(19) United States
(12) Patent Application Publication
(10) Pub. No.: US 2019/0091073 A1
(43) Pub. Date: Mar. 28, 2019

(54) DIAPER CARE SYSTEM AND METHOD FOR MULTIPLE USERS

(71) Applicant: Cvilux Corporation, New Taipei City (TW)

(72) Inventors: Ming-Hui WEN, Taichung City (TW); Wei-Nan LIU, Taipei City (TW); Sean Jeremy Wilson, Columbia, MD (US)

(21) Appl. No.: 15/896,217

(22) Filed: Feb. 14, 2018

(30) Foreign Application Priority Data
Sep. 28, 2017 (TW) 106133440

(51) Int. Cl. A61F 13/42; A61B 5/00; A61F 13/49

(52) U.S. Cl. CPC A61F 13/42; A61B 5/0022; A61F 13/49; A61B 5/7465

(57) ABSTRACT

The present disclosure provides diaper care system and method. In this method, diaper sensors senses and transmits usage parameters of diapers to a server; when it is determined that one of the diapers needs to be replaced, the server monitors the change in the usage parameter of the corresponding diaper sensor, and determines whether this diaper which needs to be replaced has been replaced or not.

### RELATED APPLICATIONS

This application claims priority to Taiwan Patent Application No. 106133440, filed Sep. 28, 2017, the entirety of which is herein incorporated by reference.

### BACKGROUND

### Field of Invention

The present invention relates to diaper care systems and methods for multiple users.

### Description of Related Art

A diaper or a nappy is a type of underwear that allows the wearer to defecate or urinate without the use of a toilet, by absorbing or containing waste products to prevent soiling of outer clothing or the external environment. Failure to change a diaper on a sufficiently regular basis can result in skin problems around the area covered by the diaper. Diapers using a very strong polymer as an absorber, and some can absorb up to 50 times its own weight of water.

However, in general care centers, diaper wearers (e.g., infants, patients, and maimed people) usually cannot directly contact with caregivers, and often have helplessness if they need to change diapers after urination.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one or more various aspects, the present disclosure is directed to diaper care systems and methods for multiple users.

An embodiment of the present disclosure is related to a diaper care system including a wireless transceiver, a server and a plurality of diaper sensors. The server is electrically connected to the wireless transceiver. The plurality of diaper sensors are configured to sense a plurality of usage parameters of the plurality of diapers respectively and to transmit the usage parameters to the wireless transceiver, so that the server determines whether the diapers need to be replaced or not, and when the diapers has a diaper which needs to be replaced as determined, the server monitors change of the usage parameter of the diaper sensor of the diaper which needs to be replaced, so as to confirm whether the diaper which needs to be replaced has been replaced or not.

In one embodiment, the diaper care system further includes a nursing device. The nursing device is configured to receive information on the diaper which needs to be replaced from the server, wherein after the nursing device sends a change completion signal to the server, the server is triggered to monitor the change of the usage parameter of the diaper sensor of the diaper which needs to be replaced, so as to confirm whether the diaper which needs to be replaced has been replaced or not.

In one embodiment, the diaper care system further includes a user device. The user device is configured to send a user experience message to the wireless transceiver, so that the server collects a time point of determining the diaper which needs to be replaced, another time point of confirming that the diaper which needs to be replaced has been changed, and the user experience information, so as to calculate a care quality score.

In one embodiment, the diaper sensor further includes a wireless transmitter, a temperature sensor, a humidity sensor and a controller. The temperature sensor is configured to sense a temperature value. The humidity sensor is configured to sense a humidity value. The controller is configured to incorporate the temperature value and the humidity value into the usage parameter so as to transmit the usage parameter to the wireless transceiver through the wireless transmitter, wherein when the temperature value and the humidity value are replaced to fall within a predetermined temperature range and a preset humidity range respectively, the server confirms that the diaper which needs to be replaced has been changed.

In one embodiment, the diaper sensor further includes a wireless transmitter, a light sensor and a controller. The light sensor is configured to sense a light wavelength. The controller is configured to incorporate the light wavelength into the usage parameter so as to transmit the usage parameter to the wireless transceiver through the wireless transmitter, wherein when the light wavelength falls within a preset spectral range, the server confirms that the diaper which needs to be replaced has been changed.

In one embodiment, the diaper sensor further includes a wireless transmitter, an accelerometer and a controller. The accelerometer is configured to sense an acceleration change. The controller is configured to incorporate the acceleration change into the usage parameter so as to transmit the usage parameter to the wireless transceiver through the wireless transmitter, wherein when the acceleration change exceeds a preset inertia range, the server confirms that the diaper which needs to be replaced has been changed.

In one embodiment, when the server determines that the diaper which needs to be replaced has not been replaced over a preset period of time, the server performs a warning function.

Another embodiment of the present disclosure is related to a diaper care method including steps of: (A) using a plurality of diaper sensors to sense a plurality of usage parameters of the plurality of diapers respectively and to transmit the usage parameters to a wireless transceiver, so that a server determines whether the diapers need to be replaced or not, wherein the server is electrically connected to the wireless transceiver; and (B) when determining that the diapers has a diaper which needs to be replaced, the server monitors change of the usage parameter of the diaper sensor of the diaper which needs to be replaced, so as to confirm whether the diaper which needs to be replaced has been replaced or not.

In one embodiment, the diaper care method further includes: using a nursing device to receive information on the diaper which needs to be replaced from the server, wherein after the nursing device sends a change completion signal to the server, the server is triggered to monitor the change of the usage parameter of the diaper sensor of the diaper which needs to be replaced, so as to confirm whether the diaper which needs to be replaced has been replaced or not.

In one embodiment, the diaper care method further includes: using a user device to send a user experience message to the wireless transceiver, so that the server collects a time point of determining the diaper which needs to be replaced, another time point of confirming that the diaper which needs to be replaced has been changed, and the user experience information, so as to calculate a care quality score.

In one embodiment, the usage parameter of the diaper which needs to be replaced comprises a temperature value sensed by a temperature sensor of the diaper sensor and a humidity value sensed by a humidity sensor of the diaper sensor, and step (B) includes: when the temperature value and the humidity value are replaced to fall within a predetermined temperature range and a preset humidity range respectively, the server confirms that the diaper which needs to be replaced has been changed.

In one embodiment, the usage parameter of the diaper which needs to be replaced comprises a light wavelength sensed by a light sensor of the diaper sensor, and step (B) includes: when the light wavelength falls within a preset spectral range, the server confirms that the diaper which needs to be replaced has been changed.

In one embodiment, the usage parameter of the diaper which needs to be replaced comprises an acceleration change sensed by an accelerometer of the diaper sensor, and step (B) includes: when the acceleration change exceeds a preset inertia range, the server confirms that the diaper which needs to be replaced has been changed.

In one embodiment, the diaper care method further includes: when the server determines that the diaper which needs to be replaced has not been replaced over a preset period of time, performing a warning function.

Technical advantages are generally achieved, by embodiments of the present invention. The present invention realizes the automatic monitoring mechanism of the multi-users diaper care and enhances the diaper wearing experience of the users.

Many of the attendant features will be more readily appreciated, as the same becomes better understood by reference to the following detailed description considered in connection with the accompanying drawings.

### BRIEF DESCRIPTION OF THE DRAWINGS

The invention can be more fully understood by reading the following detailed description of the embodiment, with reference made to the accompanying drawings as follows:

FIG. 1 is a block diagram of a diaper care system for multiple users according to some embodiments of the present disclosure;

FIG. 2 is a block diagram of a diaper sensor according to some embodiments of the present disclosure;

FIG. 3 is a flow chart of a diaper care method for multiple users according to some embodiments of the present disclosure;

FIG. 4 is a schematic diagram of changing a diaper after urination according to some embodiments of the present disclosure; and

FIG. 5 is a schematic diagram of diaper care for multiple users according to some embodiments of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

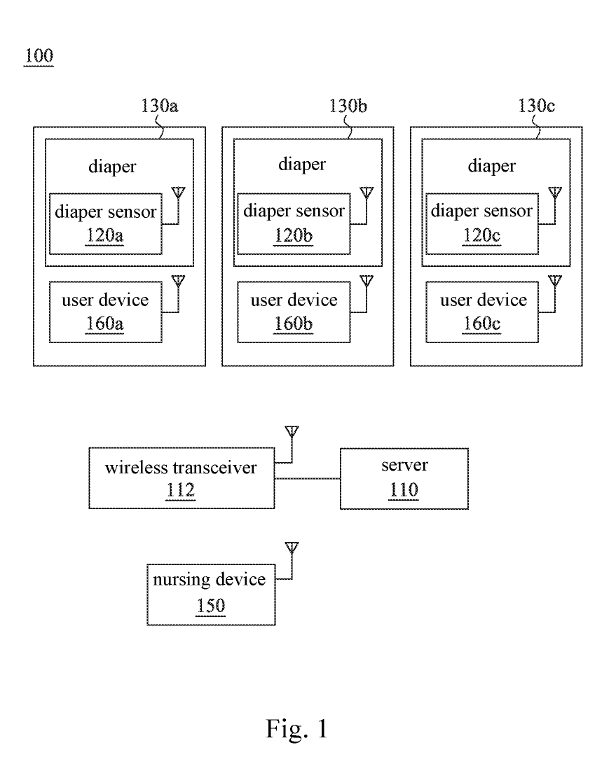

FIG. 1 is a block diagram of a diaper care system 100 for multiple users according to some embodiments of the present disclosure. As shown in FIG. 1, the diaper care system 100 includes a wireless transceiver 112, a server 110 and a plurality of diaper sensors 120a, 120b and 120c. The server 110 is electrically connected to the wireless transceiver 112. The wireless transceiver 112 can wirelessly communicate with the diaper sensors 120a, 120b and 120c. The diaper sensors 120a, 120b and 120c are detachably attached to the diaper 130a, 130b and 130c by means of a devil felt, a fastener, an adhesive, or the like, but the present invention is not limited thereto.

For example, server 110 may be a computer of a care station, the wireless transceiver 112 may be a Wi-Fi device, a Bluetooth device, or other wireless communication device, and the diapers 130a, 130b and 130c may be worn by various users (e.g., elderly under).

In use, the plurality of diaper sensors 120a, 120b and 120c are configured to sense a plurality of usage parameters of the plurality of diapers 130a, 130b and 130c respectively and to transmit the usage parameters to the wireless transceiver 112, so that the server 110 determines whether the diapers 130a, 130b and 130c need to be replaced or not, and when the diapers 130a, 130b and 130c has a diaper (e.g., diaper 130b) which needs to be replaced as determined, the server 110 monitors change of the usage parameter of the diaper sensor of the diaper 130b which needs to be replaced, so as to confirm whether the diaper 130b which needs to be replaced has been replaced or not.

In FIG. 1, the nursing device 150 may be used by a nurse. For example, the nursing device 150 may be a cell phone, a tablet, or similar electronic device. The nursing device 150 may receive a warning signal from the server 110 and notify the user with an interface, which may be an App, a webpage, a chat-bot, messages in textual pictures, voice-bot to provide messages by voice and so on.

In use, the nursing device 150 is configured to receive information on the diaper 130b which needs to be replaced from the server 110. After the nursing device 150 sends a change completion signal to the server 110, the server 110 is triggered to monitor the change of the usage parameter of the diaper sensor 120b of the diaper 130b which needs to be replaced, so as to confirm whether the diaper 130b which needs to be replaced has been replaced or not. This manner can avoid mistakenly believing that the replacement is complete while nurses do not replace the diaper 130b with a new diaper.

When the server 110 determines that the diaper 130b to be changed is not replaced, the reminder message about the diaper 130b which needs to be replaced is sent to the nursing device 150 via the wireless transceiver 112. After the nurse has replaced the diaper 130b with the new diaper, he or she attaches the diaper sensor 120b to the new diaper.

In FIG. 1, the user devices 160a, 160b and 160c can be used by various users (e.g., elderly people). For example, the user devices 160a, 160b and 160c may be cell phones, tablets, or similar electronic devices. The user devices 160a, 160b, and 160c can receive an alert signal from the server 110 and alert the user with an interface, which may be a web page, a chat-bot, a text message, a voice-bot to provide messages by voice and so on.

In use, for an instance of user device 160b, the user device 160b is configured to send a user experience message (e.g., satisfaction index, urging replacement and so on) to the wireless transceiver 112, so that the server 110 can collect a time point of determining the diaper 130b which needs to be replaced, another time point of confirming that the diaper 130b has been changed, and the user experience information, so as to calculate a care quality score.

For example, when the duration from the time point of determining the diaper 130b which needs to be replaced to the time point of confirming that the diaper 130b has been changed is shorter, the satisfaction index is higher. When receiving the urge replacement, the server 110 decreases the care quality score. When the satisfaction index is higher than a predetermined standard, the server 110 increases the care quality score.

In practice, the server 110 may calculate performance scores of nurses based on the care quality scores. On the other hand, the server 110 may also send the information about the diaper wearer to the mobile application (APP) of the mobile device of his or her family member.

When the server 110 determines that the diaper (e.g., diaper 130a) which needs to be replaced has not been replaced over a preset period of time, this situation represents that the correct position where the diaper sensor 120a is not attached or dropped, or the diaper wearers body has a condition; therefore the server 110 performs a warning function. For example, the server 110 transmits the warning message about the diaper 130a to the nursing device 150 through the wireless transceiver 112, so that the nurse can process the warning message. It should be noted that the preset period of time may be determined by the system administrator, or the server 110 may estimate the preset period for each diaper wearer according to the historical wetting interval data of each diaper wearer.

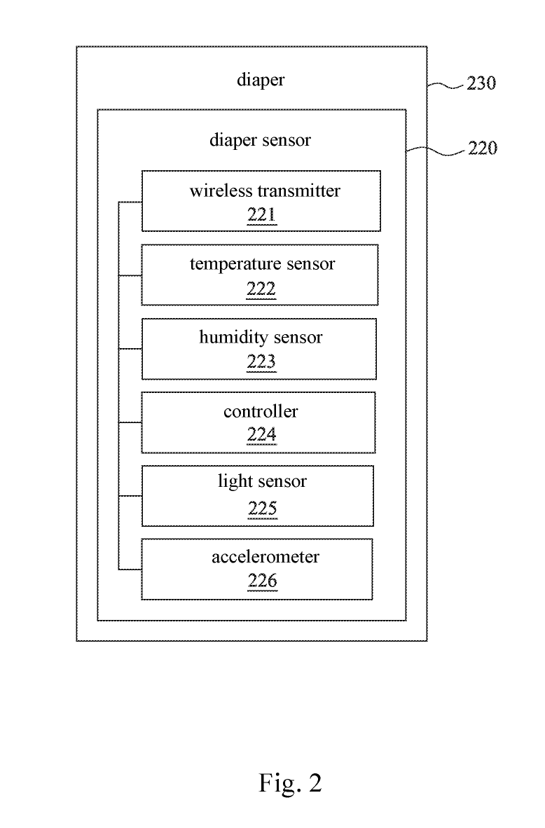

For a more complete understanding of the diaper sensor, referring FIGS. 1-2, FIG. 2 is a block diagram of a diaper sensor 220 according to some embodiments of the present disclosure. It should be noted that the diaper sensor 220 may be adapted to any of the diaper sensors 120a, 120b and 120c as described above; accordingly, the diaper 230 may be adapted to the corresponding one of the diapers 130a, 130b and 130c as described above.

In FIG. 2, the diaper sensor further includes a wireless transmitter 221, a temperature sensor 222, a humidity sensor 223, a controller 224, a light sensor 225 and an accelerometer 226. In structure, the controller 224 is electrically connected to the wireless transmitter 221, the temperature sensor 222, the humidity sensor 223, the light sensor 225 and the accelerometer 226.

In practice, the wireless transmitter 221 may include a Bluetooth module or other short-distance transmission module. The temperature sensor 222 may be an electronic component that converts temperature into electronic data. The humidity sensor 223 may be a resistive or capacitive hygrometer. The controller 224 may be a microcontroller or other circuit. The light sensor 225 may be a CCD or CMOS light sensor. The accelerometer 226 may be a gravity sensor.

In use, the temperature sensor 222 is configured to sense a temperature value of the diaper 230. The humidity sensor 223 is configured to sense a humidity value of the diaper 230. The controller 224 is configured to incorporate the temperature value and the humidity value into the usage parameter so as to transmit the usage parameter to the server 110. Accordingly, the server 110 is based on changes in temperature and humidity of the diaper 230 over time, so as to determine whether the diaper 230 needs to be replaced or not, by predicting the amount of urine and the amount and the hardness of feces.

In one embodiment, the controller 224 is configured to incorporate the temperature value and the humidity value into the usage parameter so as to transmit the usage parameter to the wireless transceiver 112 through the wireless transmitter 221. When the temperature value and the humidity value are replaced to fall within a predetermined temperature range (e.g., normal body temperature range) and a preset humidity range (e.g., atmospheric humidity range) respectively, the server 110 confirms that the diaper which needs to be replaced has been changed.

In another embodiment, the light sensor 225 is configured to sense a light wavelength. The controller 224 is configured to incorporate the light wavelength into the usage parameter so as to transmit the usage parameter to the wireless transceiver through the wireless transmitter. When the light wavelength falls within a preset spectral range (e.g., a color range of the new diaper), the server 110 confirms that the diaper which needs to be replaced has been changed.

In yet another embodiment, the accelerometer 226 is configured to sense an acceleration change. The controller 224 is configured to incorporate the acceleration change into the usage parameter so as to transmit the usage parameter to the wireless transceiver through the wireless transmitter, wherein when the acceleration change exceeds a preset inertia range (e.g., a user's general range of physical activity acceleration), the server confirms that the diaper which needs to be replaced has been changed.

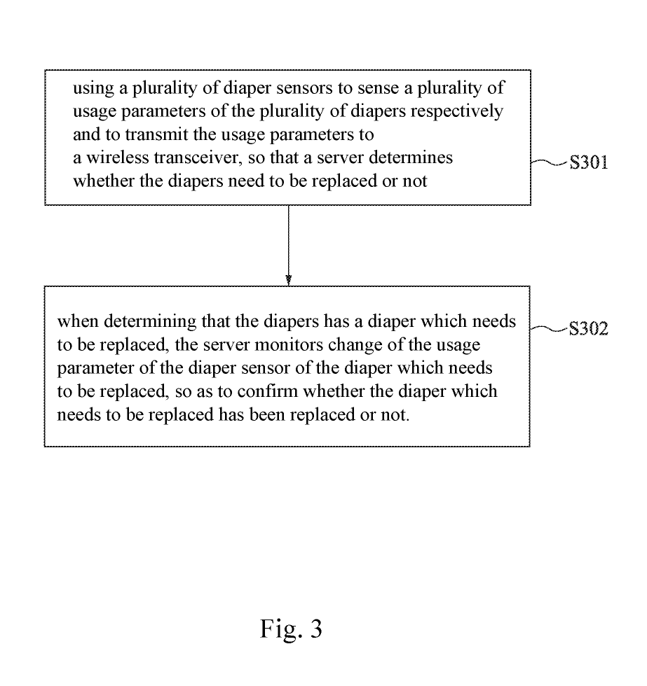

For a more complete understanding of a diaper care method performed by the diaper care system 100, referring FIG. 3, FIG. 3 is a flow chart of the diaper care method 300 according to an embodiment of the present invention. As shown in FIG. 3, the diaper care method 300 includes operations S301 and S302. However, as could be appreciated by persons having ordinary skill in the art, for the steps described in the present embodiment, the sequence in which these steps is performed, unless explicitly stated otherwise, can be altered depending on actual needs; in certain cases, all or some of these steps can be performed concurrently.

In operation S301, a plurality of diaper sensors are used 120a, 120b and 120c to sense a plurality of usage parameters of the plurality of diapers 130a, 130b and 130c respectively and to transmit the usage parameters to a wireless transceiver 112, so that a server 110 determines whether the diapers 130a, 130b and 130c need to be replaced or not, wherein the server 110 is electrically connected to the wireless transceiver 112.

In operation S302, when determining that the diapers 130a, 130b and 130c has a diaper 130b which needs to be replaced, the server 110 monitors change of the usage parameter of the diaper sensor 120b of the diaper 130b which needs to be replaced, so as to confirm whether the diaper 130b which needs to be replaced has been replaced or not.

In the diaper care method 300, a nursing device 150 is used to receive information on the diaper 130b which needs to be replaced from the server 110. After the nursing device 150 sends a change completion signal to the server 110, the server 110 is triggered to monitor the change of the usage parameter of the diaper sensor 120b of the diaper 130b which needs to be replaced, so as to confirm whether the diaper 130b which needs to be replaced has been replaced or not.

In the diaper care method 300, user devices 160a, 160b and 160c are used to send a user experience messages to the wireless transceiver 112, so that the server 110 collects a time point of determining the diaper which needs to be replaced, another time point of confirming that the diaper which needs to be replaced has been changed, and the user experience information, so as to calculate a care quality score.

In the diaper care method 300, when the server 110 determines that the diaper (e.g., diaper 130a) which needs to be replaced has not been replaced over a preset period of time, this situation represents that the correct position where the diaper sensor 120a is not attached or dropped, or the diaper wearers body has a condition; therefore the server 110 performs a warning function.

In one embodiment, the usage parameter of the diaper which needs to be replaced comprises a temperature value sensed by a temperature sensor 222 of the diaper sensor 220 and a humidity value sensed by a humidity sensor 223 of the diaper sensor 220, and operation S302 includes: when the temperature value and the humidity value are replaced to fall within a predetermined temperature range (e.g., normal body temperature range) and a preset humidity range (e.g., atmospheric humidity range) respectively, the server confirms that the diaper which needs to be replaced has been changed.

Alternatively or additionally, in one embodiment, the usage parameter of the diaper which needs to be replaced comprises a light wavelength sensed by a light sensor of the diaper sensor, and operation S302 includes: when the light wavelength falls within a preset spectral range (e.g., a color range of the new diaper), the server confirms that the diaper which needs to be replaced has been changed.

Alternatively or additionally, in one embodiment, the usage parameter of the diaper which needs to be replaced comprises an acceleration change sensed by an accelerometer of the diaper sensor, and operation S302 includes: when the acceleration change exceeds a preset inertia range (e.g., a user's general range of physical activity acceleration), the server confirms that the diaper which needs to be replaced has been changed.

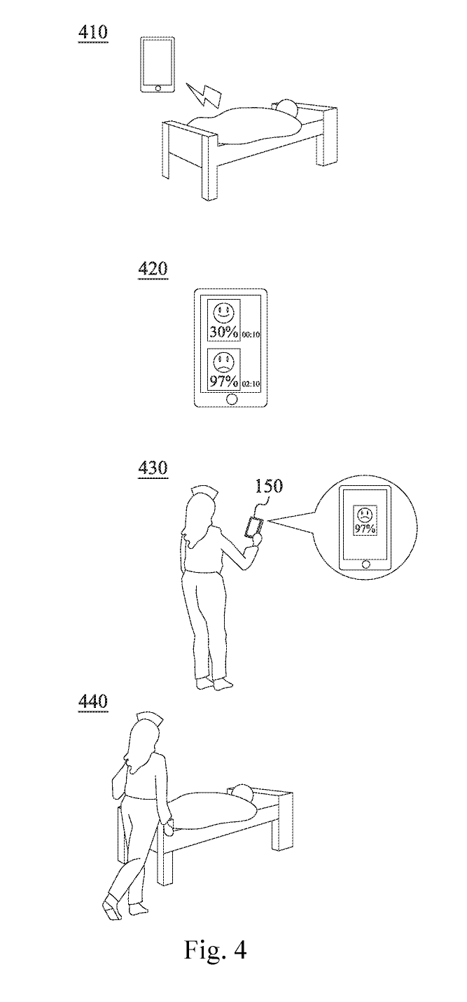

FIG. 4 is a schematic diagram of changing a diaper after urination according to some embodiments of the present disclosure. As shown in FIG. 4, in step 410, the diaper wearer excretes urine. In step 420, the diaper wearer gradually perceives that the diaper needs to be replaced. In step 430, the nurse receives the information of the diaper to be replaced through the nursing device 150. In step 440, the nurse helps the diaper wearer to change the diaper.

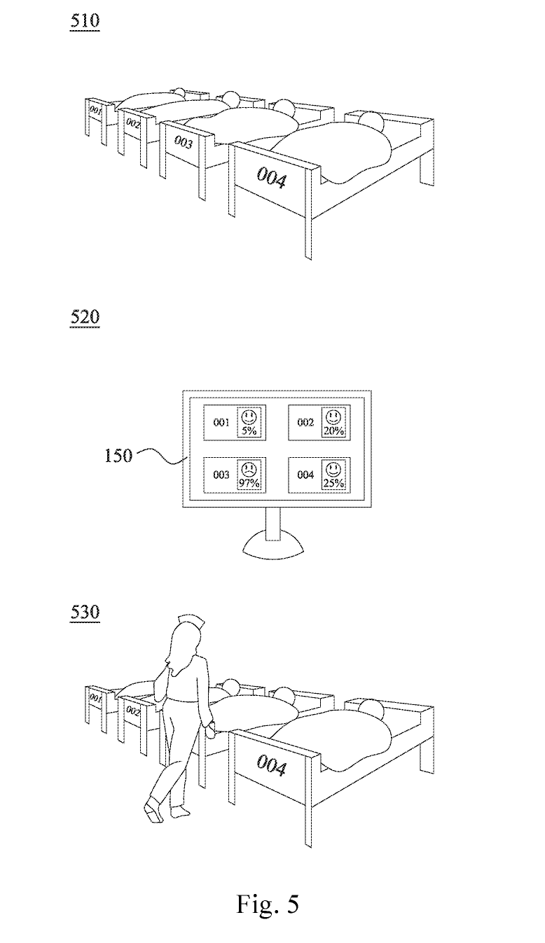

FIG. 5 is a schematic diagram of diaper care for multiple users according to some embodiments of the present disclosure. As shown in FIG. 5, in step 510, people wear diapers. In step 520, the nursing device 150 presents the information of the diaper which needs to be changed. In step 530, the nurse helps the diaper wearers to change the diaper.

In view of above, the present invention realizes the automatic monitoring mechanism of the multi-users diaper care and enhances the diaper wearing experience of the users.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A diaper care system, comprising:
a wireless transceiver;
a server electrically connected to the wireless transceiver; and
a plurality of diaper sensors configured to sense a plurality of usage parameters of the plurality of diapers respectively and to transmit the usage parameters to the wireless transceiver, so that the server determines whether the diapers need to be replaced or not, and when the diapers has a diaper which needs to be replaced as determined, the server monitors change of the usage parameter of the diaper sensor of the diaper which needs to be replaced, so as to confirm whether the diaper which needs to be replaced has been replaced or not.

2. The diaper care system of claim 1, further comprising:
a nursing device configured to receive information on the diaper which needs to be replaced from the server, wherein after the nursing device sends a change completion signal to the server, the server is triggered to monitor the change of the usage parameter of the diaper sensor of the diaper which needs to be replaced, so as to confirm whether the diaper which needs to be replaced has been replaced or not.

3. The diaper care system of claim 1, further comprising:
a user device configured to send a user experience message to the wireless transceiver, so that the server collects a time point of determining the diaper which needs to be replaced, another time point of confirming that the diaper which needs to be replaced has been changed, and the user experience information, so as to calculate a care quality score.

4. The diaper care system of claim 1, wherein the diaper sensor comprises:
a wireless transmitter;
a temperature sensor configured to sense a temperature value;
a humidity sensor configured to sense a humidity value; and
a controller configured to incorporate the temperature value and the humidity value into the usage parameter so as to transmit the usage parameter to the wireless transceiver through the wireless transmitter, wherein when the temperature value and the humidity value are replaced to fall within a predetermined temperature range and a preset humidity range respectively, the server confirms that the diaper which needs to be replaced has been changed.

5. The diaper care system of claim 1, wherein the diaper sensor comprises:
a wireless transmitter;
a light sensor configured to sense a light wavelength; and
a controller configured to incorporate the light wavelength into the usage parameter so as to transmit the usage parameter to the wireless transceiver through the wireless transmitter, wherein when the light wavelength falls within a preset spectral range, the server confirms that the diaper which needs to be replaced has been changed.

6. The diaper care system of claim 1, wherein the diaper sensor comprises:
a wireless transmitter;
an accelerometer configured to sense an acceleration change; and
a controller configured to incorporate the acceleration change into the usage parameter so as to transmit the usage parameter to the wireless transceiver through the wireless transmitter, wherein when the acceleration change exceeds a preset inertia range, the server confirms that the diaper which needs to be replaced has been changed.

7. The diaper care system of claim 1, wherein when the server determines that the diaper which needs to be replaced has not been replaced over a preset period of time, the server performs a warning function.

8. A diaper care method, comprising steps of:
(A) using a plurality of diaper sensors to sense a plurality of usage parameters of the plurality of diapers respectively and to transmit the usage parameters to a wireless transceiver, so that a server determines whether the diapers need to be replaced or not, wherein the server is electrically connected to the wireless transceiver; and
(B) when determining that the diapers has a diaper which needs to be replaced, the server monitors change of the usage parameter of the diaper sensor of the diaper which needs to be replaced, so as to confirm whether the diaper which needs to be replaced has been replaced or not.

9. The diaper care method of claim 8, further comprising:
using a nursing device to receive information on the diaper which needs to be replaced from the server, wherein after the nursing device sends a change completion signal to the server, the server is triggered to monitor the change of the usage parameter of the diaper sensor of the diaper which needs to be replaced, so as to confirm whether the diaper which needs to be replaced has been replaced or not.

10. The diaper care method of claim 8, further comprising:
using a user device to send a user experience message to the wireless transceiver, so that the server collects a time point of determining the diaper which needs to be replaced, another time point of confirming that the diaper which needs to be replaced has been changed, and the user experience information, so as to calculate a care quality score.

11. The diaper care method of claim 8, wherein the usage parameter of the diaper which needs to be replaced comprises a temperature value sensed by a temperature sensor of the diaper sensor and a humidity value sensed by a humidity sensor of the diaper sensor, and step (B) comprises:
when the temperature value and the humidity value are replaced to fall within a predetermined temperature range and a preset humidity range respectively, the server confirms that the diaper which needs to be replaced has been changed.

12. The diaper care method of claim 8, wherein the usage parameter of the diaper which needs to be replaced comprises a light wavelength sensed by a light sensor of the diaper sensor, and step (B) comprises:
when the light wavelength falls within a preset spectral range, the server confirms that the diaper which needs to be replaced has been changed.

13. The diaper care method of claim 8, wherein the usage parameter of the diaper which needs to be replaced comprises an acceleration change sensed by an accelerometer of the diaper sensor, and step (B) comprises:
when the acceleration change exceeds a preset inertia range, the server confirms that the diaper which needs to be replaced has been changed.

14. The diaper care method of claim 8, further comprising:
when the server determines that the diaper which needs to be replaced has not been replaced over a preset period of time, performing a warning function.
